(19) United States
(12) Patent Application Publication
(10) Pub. No.: US 2008/0243149 A1
(43) Pub. Date: Oct. 2, 2008

(54) Hernia Mesh Fabric For Inguinal or Hiatus Hernia Repair

(76) Inventors: Ferdinand Kockerling, Hannover (DE); Hanngorg Zimmermann, Rosstal (DE); Markus Heinlein, Gossweinstein (DE)

Correspondence Address: MCGLEW & TUTTLE, PC, P.O. BOX 9227, SCARBOROUGH STATION, SCARBOROUGH, NY, 10510-9227, US

(21) Appl. No.: 10/598,047

(22) PCT Filed: Feb. 25, 2005
(86) PCT No.: PCT/EP05/02029

§ 371 Date: Aug. 16, 2006

(30) Foreign Application Priority Data
Feb. 26, 2004 (DE) 10 2004 009 894.8

(51) Int. Cl. A61B 17/00

(52) U.S. Cl. 606/151

(57) ABSTRACT

A hernia mesh fabric for repair of in particular inguinal or hiatus hernias comprises a base sheet (1) of layered, flexible mesh material; a passage (3) in the base sheet (1) for a body canal, in particular for the spermatic cord or the oesophagus; an insertion slit (5) between the contour (4, 9) of the base sheet (1) and the passage (3) for insertion of the body canal into the passage (3); and a sewing bridge (7) which is located in the vicinity of the mouth (6) and able to be folded down on the insertion slit (5) and, on both sides thereof, to be stitched to the mesh material of the base sheet (1).

The invention relates to a hernia mesh fabric for repair of in particular inguinal or hiatus hernias, comprising the features specified in the preamble of claim 1.

Hernia meshes of the generic type are customary practice in medical engineering and standard hernia repair products. A special improvement based on that basic configuration can be taken, for example, from WO 00/67663 A1.

Hernia meshes are used for surgical repair of in particular groin hernias so as to achieve covering of the defect without stresses for stabilization of the abdominal wall. Depending on the type and position of the hernia, it can be necessary to pass a body canal through the mesh fabric, for example the spermatic cord in case of inguinal hernia or the oesophagus in case of hiatus hernia. To this end, the so-called base sheet of layered, flexible mesh material is provided with a passage. With the body canal, by nature, not having any starting end that can be threaded through the passage, the base sheet must be provided with an insertion slit between the outer contour of the base sheet and the passage for the body canal to be lead there-into.

In conventional surgery, this slit is closed after the body canal has been led into the passage by the flanks of the slit being moved into a lapping position and stitched up. However, this kind of assembly is accompanied with deformation of the hernia mesh fabric, which can negatively affect perfect positioning on the abdominal wall or on the diaphragm.

For those problems to be solved, the characterizing part of claim 1 specifies that the hernia mesh fabric, in the vicinity of the mouth of the insertion slit, is provided with a sewing bridge that is able to be folded down on the insertion slit and, on both sides thereof, to be stitched to the mesh material of the base sheet. Owing to that sewing bridge, the tabs of the hernia mesh blank that flank the insertion slit are kept level and smooth, but can be stitched up nevertheless. Any bulking and deforming of the hernia mesh fabric is avoided so that it may rest perfectly on the abdominal wall or on the diaphragm.

An especially preferred embodiment relates to the one-piece design of the sewing bridge of the mesh material of the base sheet. In this way, any complicated handling of small-surface mesh pieces for the purpose of being cut to size and fixed to the actual hernia mesh fabric can be dropped.

Further preferred configurations of a hernia mesh fabric of that kind are specified in the sub-claims. Features, details and advantages of the invention will become apparent from the ensuing description of exemplary embodiments, taken in conjunction with the drawing, in which

FIG. 1 is a plan view of a hiatus hernia mesh fabric;

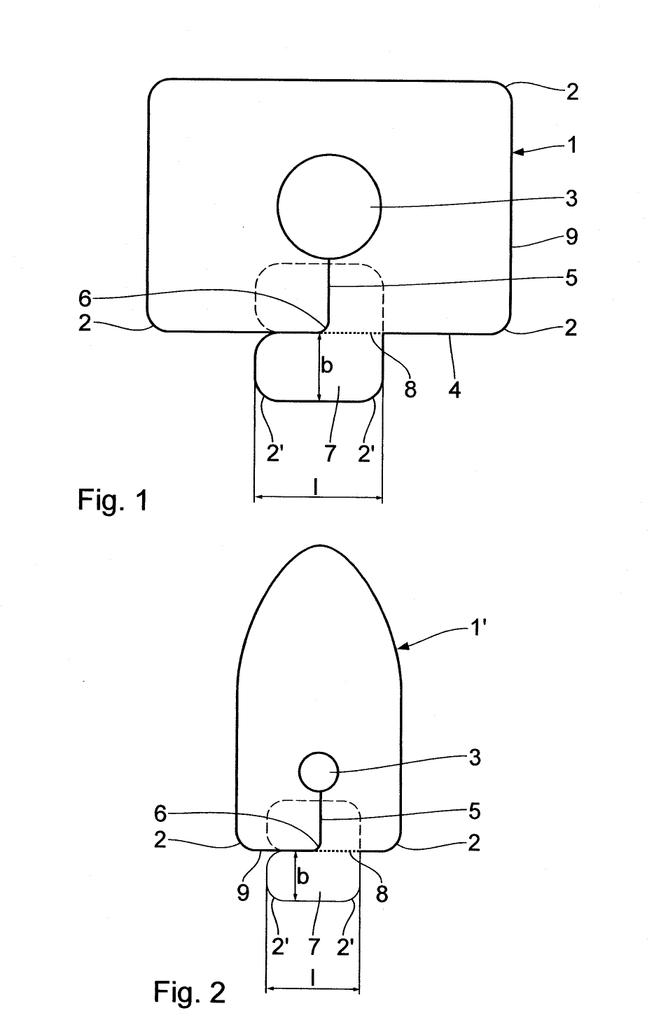

FIG. 2 is a plan view of an inguinal hernia mesh fabric;

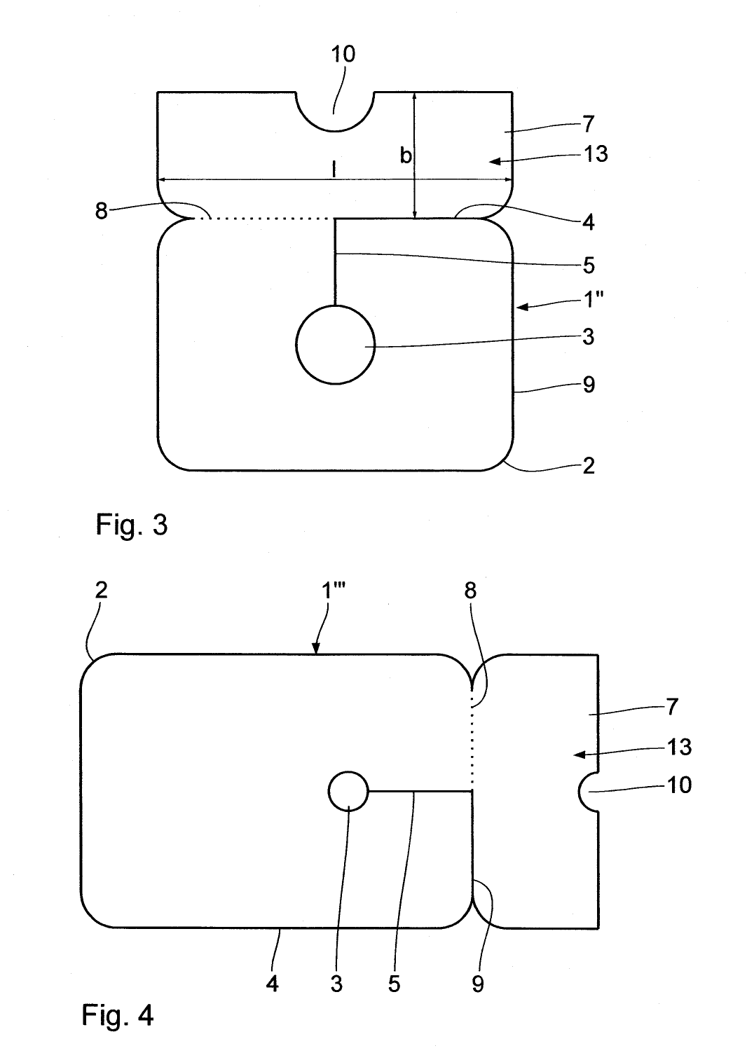

FIGS. 3 and 4 are plan views of hiatus and inguinal hernia mesh fabrics, respectively, in an alternative of FIG. 1 and FIG. 2; and

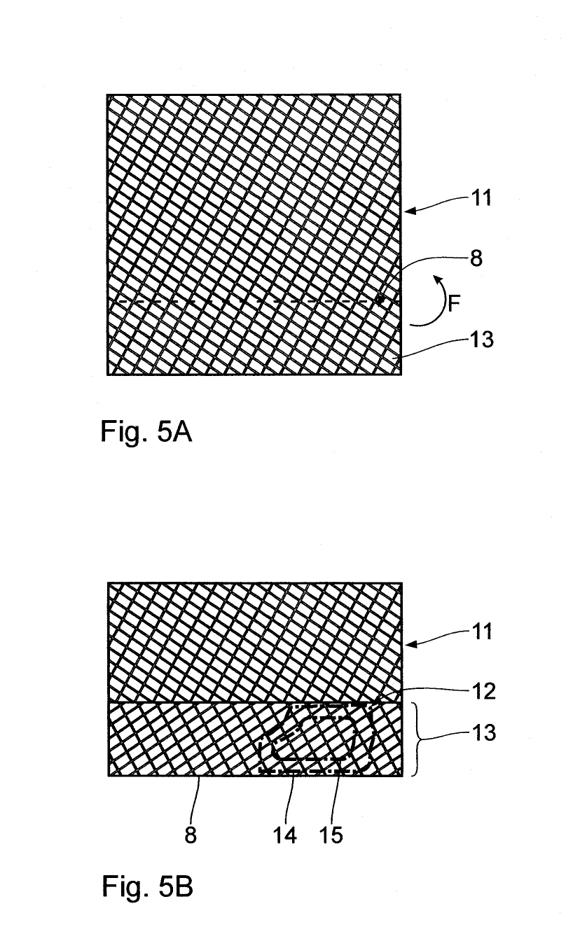

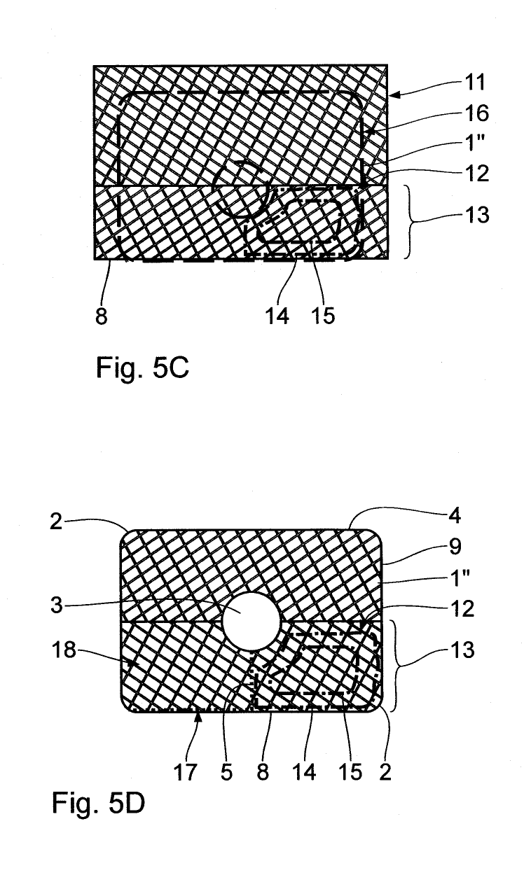

FIG. 5A-D are diagrammatic illustrations of the sequence of steps for the manufacture of the hernia mesh fabric according to FIG. 3.

The hernia mesh fabric seen in FIG. 1 serves for repair of a hiatus hernia. It comprises a base sheet 1 of customary, layered, flexible mesh material which is worked into warp-satin texture by knitting of a monofilament polypropylene thread. The grammage of the hernia mesh fabric can range between 60 and 65 g/m², but it can also be distinctly less than that.

The outer contour of the base sheet 1 is substantially rectangular with rounded corners 2. A passage 3 is arranged centrally, serving for the oesophagus to be led through in the case of the hiatus hernia mesh fabric illustrated. Proceeding from the passage 3, a straight insertion slit 5 runs toward one longitudinal side 4 of the base sheet 1, passing by a curved mouth 6 into this longitudinal side 4 in a central position. By one side of the mouth 6, an integrally attached bridge tongue 7 of substantially approximately rectangular shape is annexed to the longitudinal side 4 of the blank of the base sheet 1. The corners 2′ of the bridge tongue 7 are rounded too. The width b of the bridge tongue 7 just about corresponds to the length of the insertion slit 5, its length I approximately corresponding to twice the width b.

The shape, shown in solid lines in FIG. 1, of the base sheet 1, the passage 3, the insertion slit 5 and the sewing bridge 7 is produced by the aid of a laser cutting beam from mesh sheet material. Laser beam cutting provides for neatly melted edges, there being no risk of fiber fragments emerging from the hernia meshes.

Upon insertion, the hernia mesh fabric is passed over the oesophagus by the insertion slit 5 being opened until the oesophagus is located in the passage 3. The base sheet 1 is smoothed, with the flanks of the insertion slit 5 adjoining neatly, and then the bridge tongue 7 is doubled up inwards along the dotted folding edge 8 that is in alignment with the remaining longitudinal side 4 so that the insertion slit 5 is substantially symmetrically covered as far as slightly upstream of the passage 3 and bilaterally in the longitudinal direction. Then the bridge tongue 7 is stitched to the parts, located there-beneath, of the base sheet 1 to both sides of the insertion slit 5—not shown in detail—so that the slit 5 is stably closed.

The hernia mesh fabric seen in FIG. 2 serves for inguinal hernia repair. Its base sheet 1′ is of substantially archway shape, the passage 3 and the insertion slit 5 being arranged in the rectangular bottom part of the blank. The slit 5 mouthes into the short transverse side 9 of the base sheet 1′. By analogy to the embodiment according to FIG. 1, a bridge tongue 7 is again integrally attached to the mouth 6 of the slit 5. Once the hernia mesh fabric is positioned in such a way that the spermatic cord extends through the passage 3, the insertion slit 5 can be closed, as described in conjunction with FIG. 1, by the bridge tongue 7 being doubled up along the folding edge 8 and stitched to the bilateral areas along the flanks of the slit.

The hernia mesh fabric seen in FIG. 3 comprises a bridge tongue 7 which is greater than that of the embodiment of FIG. 1, its length corresponding to approximately the whole longitudinal side 4 of the base sheet 1″ and its width to approximately half the transverse side 9. When doubled up along the folding edge 8, the bridge tongue 7 will be located in the vicinity of the passage 3, which is compensated by the bridge tongue 7 being provided with a semicircular recess 10. Again, an insertion slit 5 is disposed in the base sheet 1″, running as far as to one end of the bridge tongue 7.

The hernia mesh fabric according to FIG. 4 comprises an enlarged bridge tongue 7 analogous to FIG. 3. In this regard, reference can be made to the description of FIG. 3 for further explanation.

Another difference from the embodiment of FIG. 2 resides in the basic shape of the base sheet 1′″, which is again substantially rectangular in this case.

The constructionally simple and convenient manufacture of the hernia mesh fabric illustrated in FIG. 3 is going to be explained, taken in conjunction with FIGS. 5A to D.

A rectangular blank 11 of the mesh material of polypropylene mentioned at the outset is taken as a starting point. In a first step, that blank is doubled up along a dashed folding line 8 (arrow F) which divides the width of the blank 11 in a ratio slightly above 2:1. (FIG. 5A).

By the aid of a polypropylene thread which corresponds to the thread material of the blank 11, a complete double-stitched seam 12 is produced on half a side of the doubled-up arrangement between the doubled-up tongue piece 13 and the mesh material, located thereunder, of the blank 11. The position of the double-stitched seam 12, which is comprised of an outer seam 14 and an inner seam 15 that is displaced inwards at a distance therefrom, is selected such that sufficient space is kept for the passage 3 and a marginal hem of the blank 11. The production of the double-stitched seam 12 is roughly outlined by a dot-dashed line in FIG. 5B.

In a subsequent manufacturing step according to FIG. 5C, the exact contour of the base sheet 1″—as mentioned above—is produced by lasering or punching from the blank 11, comprising the doubled-up tongue piece 13, as roughly outlined by a line of long dashes. Attention must be paid to the fact that the encircling, lasered or punched cutting path 16 with rounded corners and of slightly reduced dimensions as compared to the blank 1 leaves the folding edge 8 between the tongue piece 13 and the base sheet 1″ undamaged (FIG. 5C).

The hernia mesh fabric thus produced is completed by a separating cut 17 between the passage 3 and the folding edge 8 and along the folding edge 8 toward the side that is turned away from the double-stitched seam 12. That separating cut 17, roughly outlined by a dotted line in FIG. 5D, only passes through the mesh material layer that constitutes the base sheet 1″, however not through the tongue piece 13, so that the tongue-piece-13 tab, on the left of the separating cut 17 for the insertion slit 5, still adheres to the blank 11 and, after insertion of the spermatic cord or oesophagus through the insertion slit S that is formed by the separating cut 17, the tab 18 can be stitched to the part, located thereunder, of the base sheet 1″ by a surgeon.

The hernia mesh fabrics according to FIGS. 1 to 4, after being cut to size from sheet material, are provided with a continuous titanium coating that covers the entire mesh fabric surface by a prior art plasma enhanced chemical vapor deposition process. This metallization process is known, for example, from DE 199 45 299 A, providing for a titanium-containing coating of a thickness in the range of <2 μm, preferably 5 to 700 nm. Practical values of coating thickness range from 20 to 30 nm.

1. A hernia mesh fabric for repair of in particular inguinal or hiatus hernias, comprising
a base sheet of layered, flexible mesh material;
a passage in the base sheet for a body canal, in particular for a spermatic cord or a oesophagus; and
an insertion slit between a contour of the base sheet and the passage for insertion of the body canal into the passage (3); and
a sewing bridge which is located in the vicinity of a mouth of the insertion slit and which is able to be folded down on the insertion slit and, on both sides thereof, to be stitched to the mesh material of the base sheet.

2. A hernia mesh fabric according to claim 1, wherein the sewing bridge is a bridge tongue which is cut size in one piece with the mesh material of the base sheet.

3. A hernia mesh fabric according to claim 2, wherein the bridge tongue has a rectangular basic shape of such dimensioning that, when it is doubled up, the insertion slit is covered at least as far as slightly upstream of the passage.

4. A hernia mesh fabric according to claim 2, wherein the bridge tongue covers the insertion slit on both sides substantially symmetrically.

5. A hernia mesh fabric according to claim 2, wherein the bridge tongue, where directly adjoining the mouth of the insertion slit into the contour of the base sheet, is integrally attached to the base sheet have rounded corners.

6. A hernia mesh fabric according to claim 1, wherein at least one of the base sheet and the bridge tongue have rounded corners.

7. A hernia mesh fabric according to claim 1, wherein it is cut to size from meshed sheet material preferably of polypropylene by the aid of a laser cutting beam.

8. A hernia mesh fabric according to claim 1, comprising a metal-containing, continuous, biocompatible coating.

9. A hernia mesh fabric according to claim 8, wherein the coating is a titanium-containing coating of a thickness of less than 2 μm, preferably of 5 to 700 nm.

10. A hernia mesh fabric according to claim 1, wherein the bridge tongue, in a condition of pre-fabrication, is doubled up and stitched to the mesh material of the base sheet on one side of the insertion slit.

11. A hernia mesh fabric according to claim 10, wherein the unilateral stitching arrangement is a double-stitched seam, comprising an outer seam and a seam which is displaced inwards at a distance therefrom.
